(19) United States
(12) Patent Application Publication
(10) Pub. No.: US 2024/0325710 A1
(43) Pub. Date: Oct. 3, 2024

(54) Systems For Opening Welded Tubes

(71) Applicant: Terumo BCT, Inc., Lakewood, CO (US)

(72) Inventors: Dennis BEBIÉ, Denver, CO (US); James R. Ladtkow, Broomfield, CO (US); Charles L. Hake, Arvada, CO (US)

(21) Appl. No.: 18/606,015

(22) Filed: Mar. 15, 2024

Related U.S. Application Data
(60) Provisional application No. 63/455,840, filed on Mar. 30, 2023.

(51) Int. Cl. A61M 39/10; B29C 67/00

(52) U.S. Cl. CPC A61M 39/10; B29C 67/0029

(57) ABSTRACT

A tube opening device includes a body having a first side and a second side and including a tube-receiving cavity. The first side of the body includes a first belt system disposed on a first side of the tube-receiving cavity and a second belt system disposed on a second side of the tube-receiving cavity. The first and second belt systems are configured to apply pressure to a tube received in the tube-receiving cavity to open temporary seals and form a continuous tube.

### CROSS-REFERENCE TO RELATED APPLICATIONS

This application claims the benefit of U.S. Provisional Application No. 63/455,840 filed Mar. 30, 2023. The entire disclosure of the above application is incorporated herein by reference.

### FIELD

The present disclosure relates to systems and assemblies for opening welded tubes, such as tubes welded using tube welding machines.

### BACKGROUND

This section provides background information related to the present disclosure which is not necessarily prior art.

Tube welding machines (also referred to as tube welding devices) are used for connecting closed end tubes which are often connected to bags or similar containers carrying, for example, blood or blood components. Tube welding devices commonly include first and second tube-holding assemblies (e.g., first and second clamps) configured to receive first and second tubes and a space between the first and second tube-holding assemblies configured to receive a wafer (e.g., heated blade), where the tubing welding device is configured to send energy to the space such that the wafer can be heated to desired temperatures. A process used in conjunction with a tubing welding device may include sending energy to the wafer to heat the wafer to a desired temperature and urging the heated wafer into contact with each tube held by the tube-holding assemblies to temporarily seal together opposing surfaces of the respective tubes and creating molten tube ends. At least a portion of one or both of the first and second tube-holding assemblies may then be moved to align and join together the molten tube ends of the first and second tubes. The joint may be cooled and subjected to a stress to open the temporary seals providing fluid communication between the as-joined first and second tubes. Often the temporary seals are broken and the lumen opened by a technician pinching and rolling and/or squeezing and/or pulling the tubing near the joint. These manual processes are often inefficient and may cause the technician to experience hand stress and fatigue, and in certain instances, repetitive motion disorders, like carpal tunnel syndrome, trigger finger, and/or tenosynovitis. Accordingly, it would be desirable to develop systems that reduce these stressors, and methods of using the same.

### SUMMARY

This section provides a general summary of the disclosure and is not a comprehensive disclosure of its full scope or all of its features.

In various aspects, the present disclosure provides a tube opening device.

In at least one example embodiment, the tube opening device may include a body having a first side and a second side and may include a tube-receiving cavity. The first side of the body may include a first belt system and a second belt system. The first belt system may be disposed on a first side of the tube-receiving cavity. The second belt system may be disposed on a second side of the tube-receiving cavity. The first and second belt systems may be configured to apply pressure to a tube received in the tube-receiving cavity.

In at least one example embodiment, the first belt system may include a first belt that overlays at least a portion of a first belt drive and a first shaft that is in communication with the first belt drive. The first shaft may be configured to rotate the first belt drive in a first direction and also in a second direction different from the first direction.

In at least one example embodiment, the first belt system may further include a first support block disposed between the first belt drive and the tube-receiving cavity. The first support block may be configured to maintain contact between the first belt and the tube as disposed in the tube-receiving cavity between the first and second belt systems.

In at least one example embodiment, the first belt system may further include a second belt drive that is disposed on a first end of the first support block and also a third belt drive that is disposed on an opposing second end of the first support block.

In at least one example embodiment, the first shaft may be configured to be moved in the first and second directions using an automatic operation.

In at least one example embodiment, the first shaft may be configured to be moved in the first and second directions using a manual operation.

In at least one example embodiment, the second belt system may include a second belt that overlays at least a portion of a second belt drive and a second shaft that is in communication with the second belt drive. The second shaft may be configured to rotate the second belt drive in a first direction and also in a second direction different from the first direction.

In at least one example embodiment, the second belt system may further include a second support block disposed between the second belt drive and the tube-receiving cavity. The second support block may be configured to maintain contact between the second belt and the tube as disposed in the tube-receiving cavity between the first and second belt systems.

In at least one example embodiment, the second belt system may further include a third belt drive disposed on a first end of the second support block and a fourth belt drive disposed on an opposing second end of the second support block.

In at least one example embodiment, the second shaft may be configured to be moved in the first and second directions using an automatic operation.

In at least one example embodiment, the second shaft may be configured to be moved in the first and second directions using a manual operation.

In at least one example embodiment, the present disclosure provides a tube opening device. The tube opening device may include a body having a first side and a second side and may include a tube-receiving cavity. The first side of the body may include two or more gears. The second side of the body may include a first belt system that is in communication with a first gear of the two or more gears and also a second belt system that is in communication with a second gear of the two or more gears. The first belt system and the first gear may be disposed on a first side of the tube-receiving cavity. The second belt system and the second gear may be disposed on a second side of the tube-receiving cavity.

In at least one example embodiment, the first gear may include a first plurality of teeth, the second gear may include a second plurality of teeth, and the two or more gears may further include a third gear comprising a third plurality of teeth.

In at least one example embodiment, the third gear may be movable between a first position and a second position. In the first position, the third plurality of teeth may engage the first plurality of teeth of the first gear and also the second plurality of teeth of the second gear. In the second position, the third plurality of teeth do not teeth engage the first plurality of teeth of the first gear or the second plurality of teeth of the second gear.

In at least one example embodiment, the second side may further include a button configured to cause the third gear to move from the first position to the second position.

In at least one example embodiment, in the first position, movement of the first gear may be translated to the second gear and the first and second belts may be moved in opposing directions.

In at least one example embodiment, the first gear may be configured to be moved by a first drive motor.

In at least one example embodiment, in the second position, the second gear may be configured to be moved by a second drive motor.

In at least one example embodiment, the first drive motor may be configured to move the first gear in a first direction and the second drive motor may be configured to move the second gear in the first direction.

In at least one example embodiment, the first drive motor may be configured to move the first gear in a first direction and the second drive motor may be configured to move the second gear in a second direction opposing the first direction.

In at least one example embodiment, a first shaft may couple the first gear and the first belt system, and a second shaft may couple the second gear and the second belt system, where the first and second shafts travel through the body.

In at least one example embodiment, the first belt system may include one or more first pulleys and at least one of the one or more first pulleys may be coupled to the first shaft.

In at least one example embodiment, the second belt system may include one or more second pulleys and at least one of the one or more second pulleys may be coupled to the second shaft.

In at least one example embodiment, the first belt system may include one or more first pulleys and a first support block. The one or more first pulleys may include a first pulley disposed near a first end of the first support block and a second pulley disposed near a second end of the first support block. The first support block may be disposed adjacent to a first side of the tube-receiving cavity.

In at least one example embodiment, the one or more first pulleys may further include a third pulley disposed adjacent to the first support block away from the first side of the tube-receiving cavity.

In at least one example embodiment, the second belt system may include one or more second pulleys and a second support block. The one or more second pulleys may include a fourth pulley disposed near a first end of the second support block and a fifth pulley disposed near a second end of the second support block. The second support block may be disposed adjacent to a second side of the tube-receiving cavity. The second side of the tube-receiving cavity opposes the first side of the tube-receiving cavity.

In at least one example embodiment, the one or more second pulleys may further include a sixth pulley disposed adjacent to the second support block away from the second side of the tube-receiving cavity.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1A is a perspective view of an example tube welding device including a first tube-holding assembly and a second tube-holding assembly and having a tube opening device attached thereto in accordance with at least one example embodiment of the present disclosure;

FIG. 1B is a perspective view of the example tube welding device illustrated in FIG. 1A where the first and second tube-holding assemblies are in an open position in accordance with at least one example embodiment of the present disclosure;

FIG. 1C is a perspective view of the example tube welding device of FIG. 1B where the first and second tube-holding assemblies each receive a portion of a first tube and a portion of a second tube in accordance with at least one example embodiment of the present disclosure;

FIG. 1D is a simplified illustration of the movement of the first and second tube-holding assemblies as illustrated in FIGS. 1A-1C in accordance with at least one example embodiment of the present disclosure;

FIG. 2 is a cross-sectional view of a jointed tube, for example, as formed using a tube welding machine (for example, like the example tube welding machine illustrated in FIGS. 1A-1D) in accordance with at least one example embodiment of the present disclosure;

FIG. 3A is a perspective view of a first side of an example tube opening device (for example, as can be using with the example tube welding machine illustrated in FIGS. 1A-1D), which includes a first belt system and a second belt system, in accordance with at least one example embodiment of the present disclosure;

FIG. 3B is a planar view of the first side of the example tube opening device as illustrated in FIG. 3A;

FIG. 4A is a simplified illustration of example movement of first and second belt systems of an example tube opening device (for example, like the example tube opening device illustrated in FIGS. 3A-4C) where a first belt of the first belt system is moved in a first direction and a second belt of the second belt system is moved in a second direction different from the first direction to draw into a tube-receiving slot (which may also be referred to as a tube-receiving cavity) between the first and second belt systems a jointed tube in accordance with at least one example embodiment of the present disclosure;

FIG. 4B is a simplified illustration of the movement of the first and second belt systems of the example tube opening device, where the first belt of the first belt system and the second belt of the second belt system are each moved in the first direction to roll the jointed tube in accordance with at least one example embodiment of the present disclosure;

FIG. 4C is a simplified illustration of the movement of the first and second belt systems of the example tube opening device, where the first belt of the first belt system and the second belt of the second belt system are each moved in the second direction to roll the jointed tube in accordance with at least one example embodiment of the present disclosure;

FIG. 4D is a simplified illustration of the movement of the first and second belt systems of the example tube opening device, where the first belt of the first belt system is moved in the second direction and the second belt of the second belt system is moved in the first direction to eject an opened, continuous tube in accordance with at least one example embodiment of the present disclosure;

FIG. 4E is a simplified illustration of the movement of the first and second belt systems of the example tube opening device, where the first belt of the first belt system is moved in the second direction and the second belt of the second belt system is moved in the first direction such that the opened, continuous tube is ejected in accordance with at least one example embodiment of the present disclosure;

FIG. 5A is a perspective view of a second side of an example manual tube opening device (for example, as can be using with the example tube welding machine illustrated in FIGS. 1A-1D), which includes a first gear, a second gear, and a third gear, the first gear being in an engaged position, in accordance with at least one example embodiment of the present disclosure;

FIG. 5B is another perspective view of the second side of the example manual tube opening device illustrated in FIG. 5A, where the first gear is in a disengaged position in accordance with at least one example embodiment of the present disclosure; and

FIG. 5C is another perspective view of the first side of the example manual tube opening device illustrated in FIGS. 3A, 3B, 5A, and 5, where the first side includes a button for engaging or disengaging the first gear in accordance with at least one example embodiment of the present disclosure.

FIG. 5D is another perspective view of the second side of the example manual tube opening device illustrated in FIG. 5A, where the first gear is in communication with a first drive motor and the second gear is in communication with a second drive motor.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Example embodiments will now be described more fully with reference to the accompanying drawings.

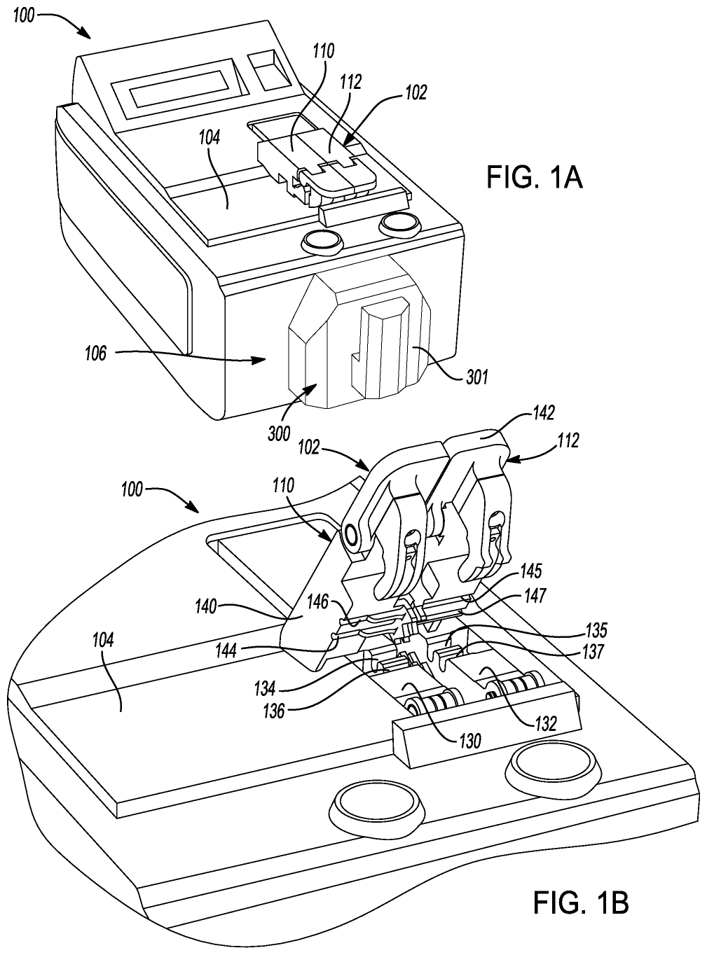

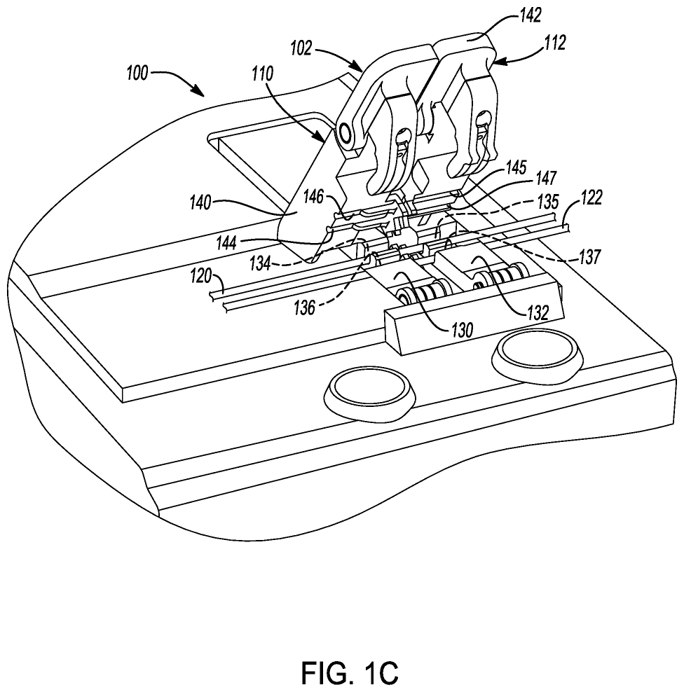

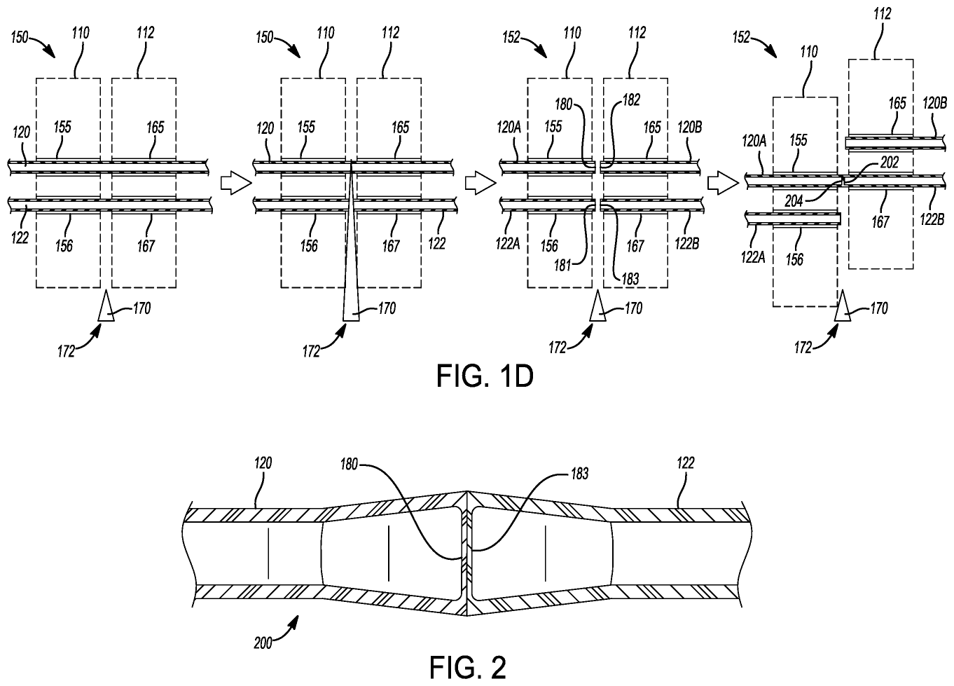

An example tube welding device (also referred to as a tube-joining machine) 100 is illustrated in FIGS. 1A-1D. The tube welding device 100 includes a tube clamp arrangement 102 that is configured to receive at least a portion of a first tube 120 and at least a portion of a second tube 122. The first and second tubes 120, 122 may be connected to bags or similar containers carrying, for example, blood or blood components. In at least one example embodiment, the tube clamp arrangement 102 includes a first tube-holding assembly (which may also be referred to as a first tube-receiving assembly) 110 adjacent to a second tube-holding assembly (also may also be referred to as a second tube-receiving assembly) 112. In at least one example embodiment, the first tube-holding assembly 110 is a first tube-holding clamp, and the second tube-holding assembly 112 is a second tube-holding clamp.

In at least one example embodiment, the first tube-holding assembly 110 may include a first or bottom clamp portion (which may also be referred to as a bottom portion) 130 and a second or top clamp portion (which may also be referred to as a top portion) 140. The bottom clamp portion 130 of the first tube-holding assembly 110 may be movably coupled to the top clamp portion 140 of the first tube-holding assembly 110, such that the first tube-holding assembly 110 may move between a closed position to an open position via the coupling. For example, in at least one example embodiment, the bottom clamp portion 130 of the first tube-holding assembly 110 may be fixedly secured to a major plane 104 of the tube welding device 100 and a top clamp portion 140 may be movable between a first (or closed) clamp state as illustrated, for example, in FIG. 1A and a second (or open) clamp state as illustrated, for example, in FIG. 1B. In at least one example embodiment, The bottom clamp portion 130 of the first tube-holding assembly 110 may be hingedly coupled to the top clamp portion 140 of the first tube-holding assembly 110.

Similarly, in at least one example embodiment, the second tube-holding assembly 112 may include a first or bottom clamp portion (which may also be referred to as a bottom portion) 132 and a second or top clamp portion (which may also be referred to as a top portion) 142. The bottom clamp portion 132 of the second tube-holding assembly 112 may be movably coupled to the top clamp portion 142 of the second tube-holding assembly 112, such that the second tube-holding assembly 112 may move between a closed position to an open position via the coupling. For example, in at least one example embodiment, the bottom clamp portion 132 of the second tube-holding assembly 112 may be fixedly secured to a major surface 104 of the tube welding device 100 and a top clamp portion 142 may be movable between a first (or closed) clamp state as illustrated in FIG. 1A and a second (or open) clamp state as illustrated in FIG. 1B. In at least one example embodiment, the bottom clamp portion 132 of the second tube-holding assembly 112 may be hingedly coupled to the top clamp portion 142 of the second tube-holding assembly 112.

The first and second tube-holding assemblies 110, 112 are each configured to receive at least a portion of first and second tubes 120, 122. In at least one example embodiment, for example as illustrated in FIGS. 1B and 1C, the bottom clamp portion 130 of the first tube-holding assembly 110 may include a first tube-receiving crevice or recess or surface 134 configured to receive or engage a first portion of the first tube 120 and a second tube-receiving crevice or recess or surface 136 configured to receive or engage a first portion of the second tube 122. Similarly, the top clamp portion 140 of the first tube-holding assembly 110 may include a first tube-receiving crevice or recess or surface 144 also configured to receive or engage the first portion of the first tube 120 and a second tube-receiving crevice or recess or surface 146 also configured to receive or engage the first portion of the second tube 122. For example, the first tube-receiving crevice 134 of the bottom clamp portion 130 of the first tube-holding assembly 110 may align with the first tube-receiving crevice 144 of the top clamp portion 140 of the first tube-holding assembly 110 to form a first tube-receiving cavity 155 (when the first tube-holding assembly 110 is in a closed state, as illustrated, for example, in FIG. 1A). Likewise, the second tube-receiving crevice 136 of the bottom clamp portion 130 of the first tube-holding assembly 110 may align with the second tube-receiving crevice 146 of the bottom clamp portion 130 of the first tube-holding assembly 110 to form a second tube-receiving cavity 156 (when the first tube-holding assembly 110 is in a closed state, as illustrated, for example, in FIG. 1A).

Like the bottom clamp portion 130 of the first tube-holding assembly 110, the bottom clamp portion 132 of the second tube-holding assembly 112 may include a first tube-receiving crevice or recess or surface 135 configured to receive or engage a second portion of the first tube 120 and a second tube-receiving crevice or recess or surface 137 configured to receive or engage a second portion of the second tube 122. Like the top clamp portion 140 of the first tube-holding assembly 110, the top clamp portion 142 of the second tube-holding assembly 112 may include a first tube-receiving crevice or recess or surface 145 also configured to receive or engage the second portion of the first tube 120 and a second tube-receiving crevice or recess or surface 147 also configured to receive or engage the second portion of the second tube 122. For example, the first tube-receiving crevice 135 of the bottom clamp portion 132 of the second tube-holding assembly 112 may align with the first tube-receiving crevice 145 of the top clamp portion 142 of the second tube-holding assembly 112 to form a third tube-receiving cavity 165 (when the second tube-holding assembly 112 is in a closed state, as illustrated, for example, in FIG. 1A). Likewise, the second tube-receiving crevice 137 of the bottom clamp portion 132 of the second tube-holding assembly 112 may align with the second tube-receiving crevice 147 of the top clamp portion 142 of the second tube-holding assembly 112 to form a fourth tube-receiving cavity 167 (when the second tube-holding assembly 112 is in a closed state, as illustrated, for example, in FIG. 1A).

The tube welding device 100 may be configured to move one or more portions of the first tube-holding assembly 110 and/or one or more portions of the second tube-holding assembly 112. In at least one example embodiment, the tube welding device 100 may include one or more motorized mechanisms for moving the one or more portions of the first tube-holding assembly 110 and/or one or more portions of the second tube-holding assembly 112. For example, as detailed in the Atty. Docket No. 18955-000205-US-PS1, titled AUTOMATIC WELD/RESET MOTION OF CLAMPS AFTER CLAMP CLOSING/OPENING AND TUBE REMOVAL and listing James Ladtkow as inventor, filed the same day hereto and assigned U.S. App. No. 63/455,873, the entire contents of which are herein incorporated by reference.

In at least one example embodiment, the one or more portions of the first tube-holding assembly 110 and/or the one or more portions of the second tube-holding assembly 112 may be movable in a first direction along a major axis of the tube welding machine 100 and/or a second direction along a minor axis of the tube welding machine 100. In at least one example embodiment, for example as illustrated FIG. 1D, the one or more portions of the first tube-holding assembly 110 and one or more portions of the second tube-holding assembly 112 may both be in a first or initial position 150 such that the first tube-receiving cavity 155 of the first tube-holding assembly 110 is aligned with the third tube-receiving cavity 165 of the second tube-holding assembly 112 and the second tube-receiving cavity 156 of the first tube-holding assembly 110 is aligned with the fourth tube-receiving cavity 167 of the second tube-holding assembly 112.

In the initial position 150, the first tube-receiving cavity 155 of the first tube-holding assembly 110 and the third tube-receiving cavity 165 of the second tube-holding assembly 112 aligned therewith may receive adjoining portions of the first tube 120, and the second tube-receiving cavity 156 of the first tube-holding assembly 110 and the fourth tube-receiving cavity 167 of the second tube-holding assembly 112 aligned therewith may receive adjoining portions of the second tube 122, as illustrated for example, in FIGS. 1C and 1D. In at least one example embodiment, the one or more portions of the first tube-holding assembly 110 may be movable relative to the one or more portions of the second tube-holding assembly 112 and/or the one or more portions of the second tube-holding assembly 112 may be movable relative to the one or more portions of the first tube-holding assembly 114, such that, as illustrated in FIG. 1D, a second position 152 is defined, where the first tube-receiving cavity 155 of the first tube-holding assembly 110 is aligned with the third tube-receiving cavity 165 of the second tube-holding assembly 112.

In at least one example embodiment, the tube welding device 100 is configured to receive a wafer 170 within a gap or space between the first tube-holding assembly 110 and the second tube-holding assembly 112. In at least one example embodiment, for example, as illustrated best in FIG. 1D, when the first tube-holding assembly 110 and the second tube-holding assembly 112 are aligned in the initial position 150, the tube welding device 100 may be configured to send energy to the wafer 170 to heat the wafer to a desired temperature (i.e., a tube-joining temperature) and to move the heated wafer 170 from a first non-contact position 172 to a second contact position 174 where the wafer 170 contacts the first and second tubes 120, 122 to temporarily seal together opposing surface of the respective tubes 120, 122 while splitting the tubes into first and second halves 120A, 120B, 122A, 122B and creating molten tube ends (including, for example, first and second molten tube ends 180, 182 for the first tube 120 and first and second molten tube ends 181, 183 for the second tube 122). For example, the heated wafer 170 may cause the portions of the tubes 120, 122 contacted to melt. In at least one example embodiment, the tube-joining temperature may be determined as detailed in the Atty. Docket No. 18955-000211-US-PS1, titled AUTOMATIC POWER ADJUSTMENTS BASED ON TUBING STATE DETECTION FOR TUBE WELDING DEVICES and listing Jesse Janzen, Jeremy Kolenbrander, and James R. Ladtkow as inventors, filed the same day hereto and assigned U.S. App. No. 63/455,817, the entire contents of which are herein incorporated by reference.

In at least one example embodiment, the wafer 170 may be heated using a radiant or conductive heat source the is configured to heat the wafer 170 based on instructions received from a controller. Alternatively, or additionally, the wafer 170 may include an embedded resistive heating element (not shown) and the tube-joining device 100 may be configured to supply an electric current to the embedded resistive heating element based on an instruction received from the controller. In at least one example embodiment, the wafer 170 includes a conductive material, like copper. In at least one example embodiment, the wafer 170 may be a replaceable wafer. For example, the wafer 170 may be removed and replaced after selected use.

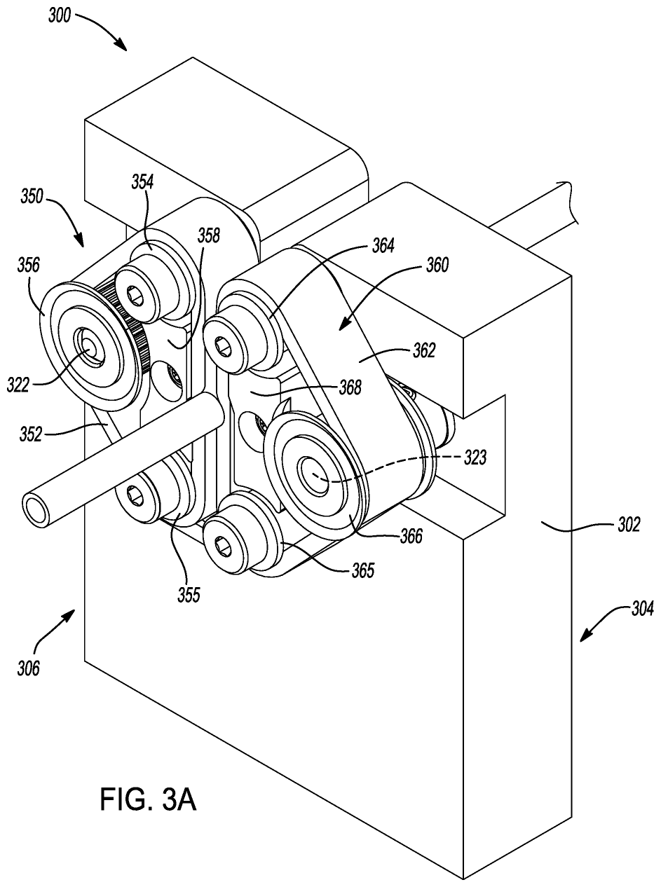

In at least one example embodiment, the tube welding device 100 may include an actuator that is in communication with the controller and that is configured to move the wafer 170 between the first non-contact position 172 and the second contact position 174. After the heated wafer 170 is contacted to the first and/or second tubes 120, 122 (i.e., after the welding event and formation of the molten tube ends 180, 181, 182, 183), the wafer 170 may be displaced—for example, returned to its first non-contact position 172 (i.e., retracted)—and the tube welding device 100, as discussed above, may be configured to move the one or more portions of the first tube-holding assembly 110 and/or the one or more portions of the second tube-holding assembly 112 to the second position 152 such that the first molten tube end 180 of the first tube 120 is brought into contact with the second molten tube end 183 of the second tube 122 (and/or the second molten tube end 183 of the second tube 122 is brought into contact with the first molten 180 of the first tube 120) to form a jointed tube 200, as illustrated, for example, in FIG. 2. In at least one example embodiment, the jointed tube 200 may be cooled (for example, passively to room temperature, which is often between about 20° C. and about 22° C.) and subjected to a stress that breaks the temporary seals of the molten tube ends 180, 183 opening the lumen 210 providing fluid communication between the as-joined first and second tubes 120, 122.

In at least one example embodiment, the tube welding device 100 may include a tube opening device (also referred to as a tube opener) 300 that is configured to break the temporary seals of the molten tube ends 180, 183 opening the lumen 210 to provide fluid communication between the as-joined first and second tubes 120, 122. For example, as illustrated, in at least one example embodiment, the tube opening device 300 may be fixedly or removably secured to a first side 106 of the tube welding device 100. Although the tube opening device 300 is illustrated as being disposed on a first side 106 of the tube welding device 100, it should be appreciated that, in other example embodiments, the tube opening device 300 may be disposed on one or more other sides of the tube welding device 100 or not attached or coupled to the tube welding device 100.

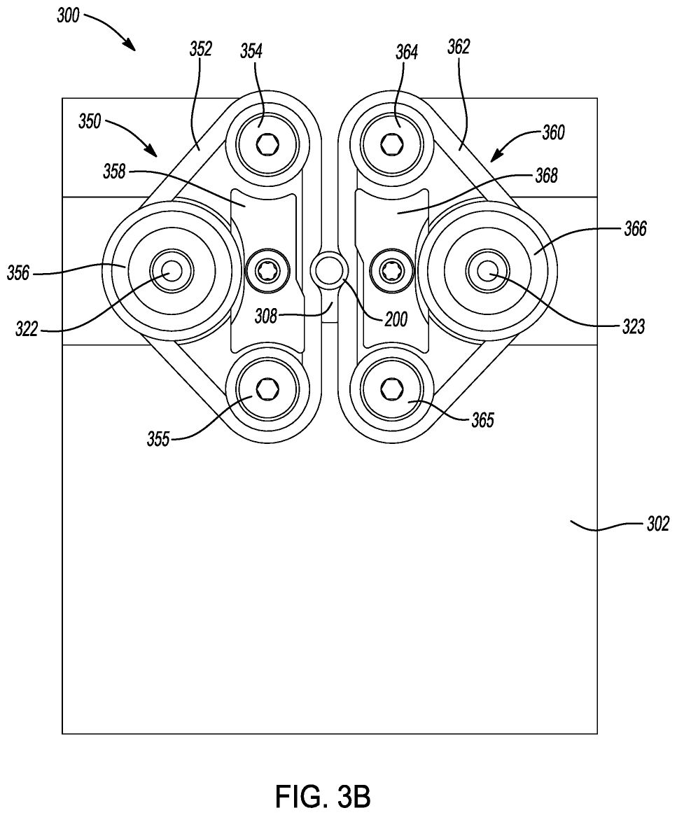

In at least one example embodiment, for example, as illustrated in FIGS. 3A-3F, the tube opening device 300 may include a body 302 having a first side 306 and a second side 304 and a tube-receiving slot (which may also be referred to as a tube-receiving cavity) 308 defined in the body 302 that extends from the first side 306 to the second side 304 and that is configured to receive the jointed tube 200. In at least one example embodiment, the tube opening device 300 may include a housing 301 that encases at least a portion of the body 302. The housing 301 may be attached to the tube welding device 100, as illustrated in FIG. 1A.

In at least one example embodiment, the first side 306 of the body 302 includes a first belt system 350 and a second belt system 360. The first belt system 350 may be disposed on a first side of the tube-receiving slot 308, while the second belt system 360 may be disposed on a second side of the tube-receiving slot 308. That is, the second belt system 360 may be substantially parallel with the first belt system 350. The first and second belt systems 350, 360 each include a belt 352, 362 driven by one or more cogged pulleys 354, 355, 356, 364, 365, 366.

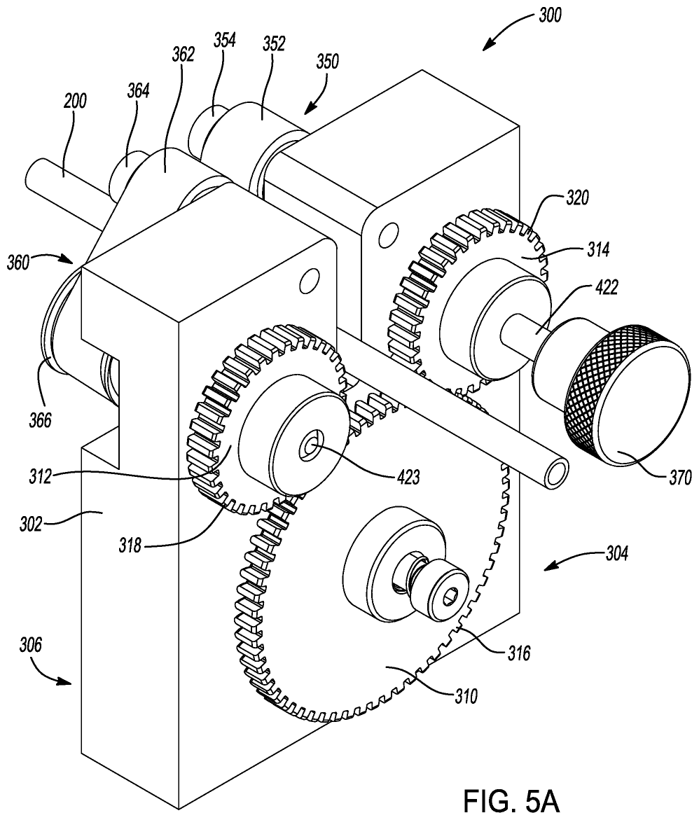

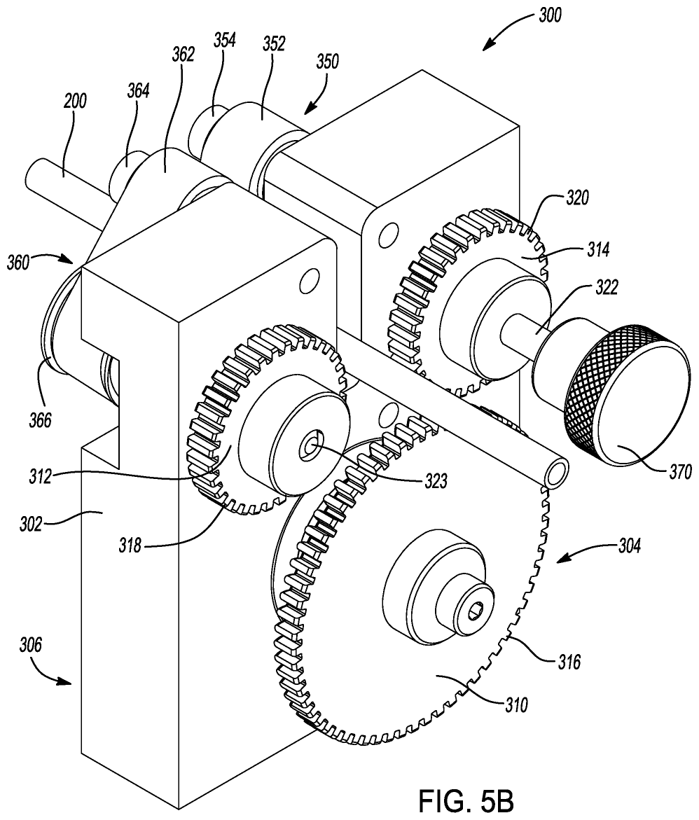

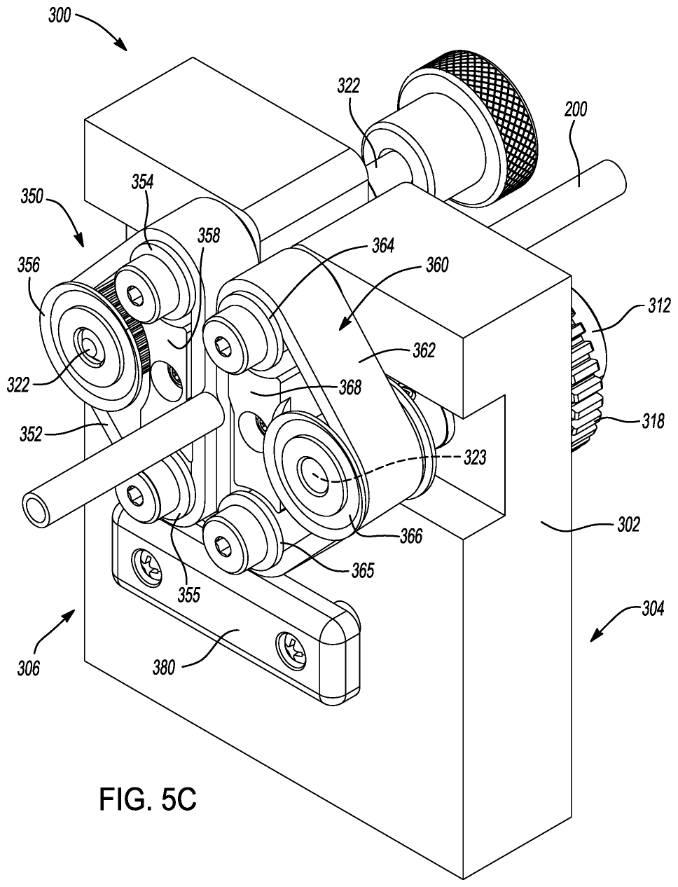

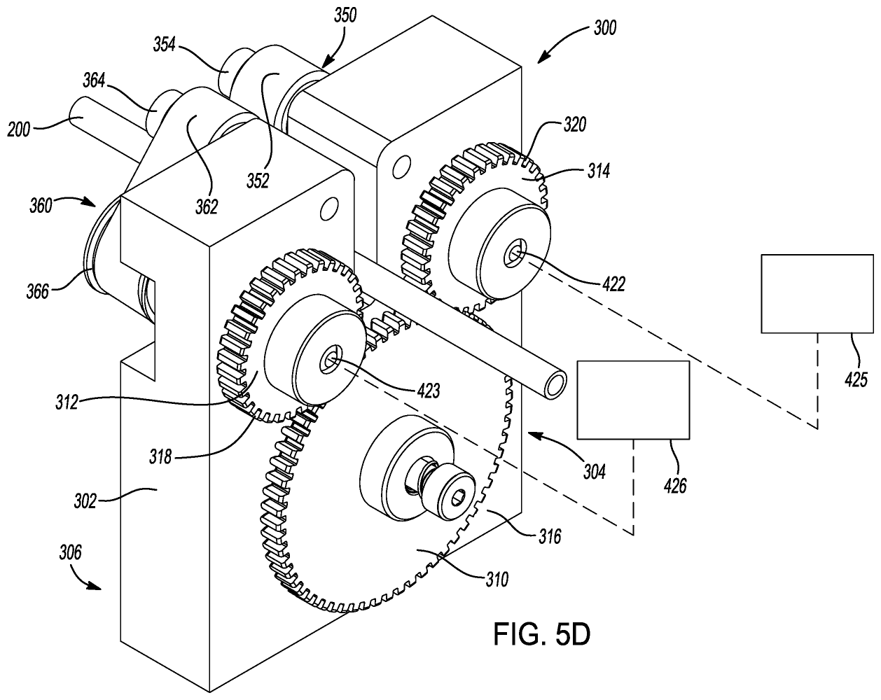

In at least one example embodiment, the first belt system 350 includes a first cogged pulley or belt drive 354, a second cogged pulley or belt drive 355, and a third cogged pulley or belt drive 356 over which a first belt 352 moves. The first, second, and third belt drives 354, 355, 356 of the first belt system 350 may be secured to the first side 306 of the body 302, such that the third belt drive 356 when activated may rotate in a clockwise or counterclockwise direction causing the first and second belt drives 354, 356 to also rotate. For example, in at least one example embodiment, tube opening device 300 may include a first shaft 322 that is in communication with the third belt drive 356 of the first belt system 350. The first shaft 322 may be manually or automatically engaged. For example, in at least one example embodiment, as illustrated in FIGS. 5A-5C, the first shaft 322 may be rotated clockwise or counter-clockwise clockwise or counter-clockwise manually using a handle 370 or the like. In at least one example embodiment, as illustrated in FIG. 5D, the first shaft 322 may be rotated clockwise or counter-clockwise clockwise or counter-clockwise using a first drive motor or other actuator 425. In each instance, movement of the first shaft 322 may engage or activate the first belt system 350. More particularly, movement of the first shaft 322 and therefore the third belt drive 356 may cause the first belt 352 to move across and around the first belt drive 354 and also the second belt drive 355 of the first belt system 350. In this manner, the third belt drive 356 of the first belt system 350 may be a conventional or common belt drive, while the first and second belt drives 364, 365 are actually conventional or common pulleys.

In at least one example embodiment, the first belt system 350 may also include a first support block 358. For example, the first support block 358 may extend between and physically separate the first and second cogged pulleys 354, 355 of the first belt system 350. That is, the first and second cogged pulleys 354, 355 may be disposed near or adjacent to opposing ends of the first support block 358. The third cogged pulley 356 of the first belt system 350 may be disposed adjacent to a lateral side of the first support block 358. The first support block 358 of the first belt system 350 may be configured to exert a pressure on the first belt 352 (for example, at least via its relationship with the first and second cogged pulleys 354, 355) to help ensure and maintain contact between the first belt 352 and the jointed tube 200 as disposed in the tube-receiving slot 308 between the first and second belt systems 350, 360. Although illustrated as having a generally rectangular shape, it should be appreciated that, in other example embodiments, the first support block 358 of the first belt system 350 may take a variety of shapes and configurations.

In at least one example embodiment, like the first belt system 350, the second belt system 360 may include a first cogged pulley or belt drive 364, a second cogged pulley or belt drive 365, and a third cogged pulley or belt drive 366. The first, second, and third belt drives 364, 365, 366 of the second belt system 360 may be secured to the first side 306 of the body 302, such that the third belt drive 366 when activated may rotate in a clockwise or counterclockwise direction causing the first and second belt drives 364, 365 to also rotate. For example, in at least one example embodiment, the tube opening device 300 may include a second shaft 323 that is in communication with the third belt drive 366 of the second belt system 360. The second shaft 323 may be manually or automatically engaged. For example, in at least one example embodiment, as illustrated in FIG. 5D, the second shaft 323 may be rotated in clockwise or counterclockwise clockwise or counter-clockwise using a second drive motor or other actuator 426. Movement of the second shaft 323 may engage or activate the second belt system 350. More particularly, movement of the second shaft 323 may cause the second belt 362 to move across and around the first belt drive 364 and also the second belt drive 365 of the second belt system 360. In this manner, the third belt drive 366 of the second belt system 360 may be a conventional or common belt drive, while the first and second belt drives 364, 365 are actually conventional or common pulleys.

In at least one example embodiment, like the first belt system 350, the second belt system 360 may also include a second support block 368. For example, the second support block 368 may extend between and physically separate the first and second cogged pulleys 364, 365 of the second belt system 360. That is, the first and second cogged pulleys 364, 365 may be disposed near or adjacent to opposing ends of the second support block 368. The third cogged pulley 366 of the second belt system 360 may be disposed adjacent to a lateral side of the support block 368. The second support block of the second belt system 360 may be configured to exert a pressure on the second belt 362 (for example, at least via its relationship with the first and second cogged pulleys 364, 365) to help ensure and maintain contact between the second belt 362 and the jointed tube 200 as disposed in the tube-receiving slot 308 between the first and second belt systems 350, 360. Although illustrated as having a generally rectangular shape, it should be appreciated that, in other example embodiments, the second support block 368 of the second belt system 360 may take a variety of shapes and configurations.

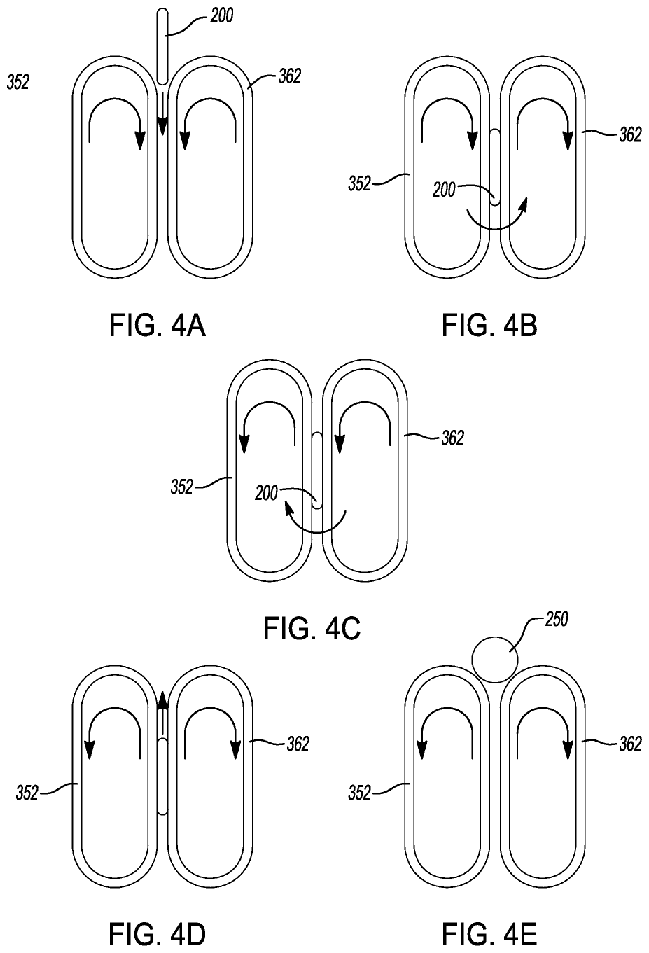

The pressure asserted by the first and second belt systems 350, 360 on the jointed tube 200 mimics the rolling pressure as previously applied by an operator. For example, in at least one example embodiment, a clockwise rotation (as view from the first side 306 of the tube opening device 300) of the first shaft 322 and therefore the first belt 352 (i.e., movement of the first shaft 322 and the first belt 352 in a first direction) and a counter-clockwise rotation as view from the first side 306 of the tube opening device 300) of the second shaft 323 and therefore the second belt 362 (i.e., movement of the second shaft 323 and the second belt 363 in a second direction)—in other words, rotation of the first and second belts 352, 362 in opposing first and second directions (i.e., a feed state)—can work to draw the jointed tube 200 into the tube-receiving slot 308 as illustrated in FIG. 4A.

Once the jointed tube 200 is positioned in the tube-receiving slot 308, at least one of a first and second rolling state may be engaged to break the temporary seals of the molten tube ends 180, 183 opening the lumen 210 providing fluid communication between the as-joined first and second tubes 120, 122. As illustrated in FIG. 4B, in the first rolling state, the first shaft 322 and therefore the first belt 352 move in the clockwise or first direction, and the second shaft 332 and therefore the second belt 362 move in the same clockwise or first direction. In other words, in the first rolling state, the first shaft 322 and the first belt 352 and the second shaft 332 and the second belt 362 may be moved in the same direction. As illustrated in FIG. 4C, in the second tolling state, the first shaft 322 and therefore the first belt 352 move in the counterclockwise or second direction, and the second shaft 332 and therefore the second belt 362 move in the same counter clockwise or second direction. In other words, in the second rolling state, the first shaft 322 and the first belt 352 and the second shaft 332 and the second belt 362 may be moved in the same direction. In at least one example embodiment, an opening sequence may include one or more rolling state events, including, for example, the first rolling state followed by the second rolling state, or the second rolling state followed by the first rolling state, or the first rolling state followed by the second rolling state followed again by the first rolling state, or the like.

Once the temporary seals are broken and the lumen 210 is open, the first and second shafts 322, 323 may be moved to eject the opened, continuous tube 250 from the tube-receiving slot 308. For example, as illustrated in FIGS. 4D and 4E, the first shaft 322 and therefore the first belt 352 may be rotated in the counterclockwise or second direction, while the second shaft 323 and therefore the second belt 362 are moved in the clockwise or first direction. That is, the first shaft 322 and the first belt 352 are moved in a direction different from the second shaft 323 and the second belt 362.

As discussed above, the first and second shafts 322, 323 may be manually or automatically engaged. In at least one example embodiment, the second side 304 of the body 302 may include two or more gears 310, 312, 314 where one or more of the gears 310, 312, 314 is configured to be manually adjusted using, for example, a handle 370, as illustrated in FIGS. 5A-5C. Although a handle 370 is illustrated in FIGS. 5A-5C, it should be appreciated that, in other example embodiments (see, e.g., FIG. 5D), one or more motors or actuators may be used in place of the illustrated handle.

In at least one example embodiment, the first side 304 of the body 302 may include a first gear 310, a second gear 312, and a third gear 314. The first gear 310 may be disposed below the tube-receiving cavity 308, while the second and third gears 312, 314 may be disposed on either side of the tube-receiving cavity 308. The first gear 310 may have a first average diameter that is larger than a second average diameter of the second gear 312 and also a third average diameter of the third gear 314. The second average diameter of the second gear 312 may be the same as the third average diameter of the third gear.

In at least one example embodiment, the first gear 310 may be movable between a first or engaged position (for example, as illustrated, in FIG. 5A) and a second or disengaged position (for example, as illustrated, in FIG. 5B). In at least one example embodiment, as illustrated in FIG. 5C, the first side 306 of the body 302 may include a button 380 that can be used to engage or disengage the first gear 310. Each of the gears 310, 312, 314 may have a general disk shape with a plurality of teeth disposed along an outer perimeter. The first gear 310 may include a first plurality of teeth 316. In the engaged position, as illustrated in FIG. 5A, the first plurality of teeth 316 of the first gear 310 may be configured to engage with a second plurality of teeth 318 of the second gear 312 and also a third plurality of teeth 320 of the third gear 314 a rotative force as applied to one of the three gears will cause related movement for the other two gears. For example, the third gear 314 may be rotated using a first shaft 422 connected thereto and the movement of the third gear 314 may be translated to the first gear 310 and also the second gear 312. The first shaft 422 may be manually engaged using the handle 370. In contrast, when the first gear 310 is in the disengage position as illustrated in FIG. 5B, the first plurality of teeth 316 of the first gear 310 will not engage with the second plurality of teeth 318 of the second gear 312 or the third plurality of teeth 320 of the third gear 314 and rotation of the first shaft 422 and the third gear 314 in communication therewith will not translate to the first gear 310 or the second gear 312.

The first shaft 422 and the third gear 314 may also be connected to the third cogged pulley 356 of the first belt system 350 such that movement of the third gear 314 engages the first belt system 350. Similarly, a second shaft 423 may be connected to both the second gear 312 and the third cogged pulley 366 of the second belt system 360 such that movement of the second gear 312 engages the second belt system 360. When the first gear 210 is in the engaged position, motion of the first shaft 422 and the third gear 314 in communication therewith can be translated to the second gear 314 and therefore the third cogged pulley 366 of the second belt system 360. In contrast, when the first gear 210 is in the disengaged position, the motion of the first shaft 422 and the third gear 314 in communication therewith is not translated to the second gear 314, rather the second gear 312 may be moved independently of the first and third gears 310, 312. Like the first shaft 422 and the third gear 314 in communication therewith, the second shaft 423 and the second gear 312 in communication therewith may be manually or automatically engaged, for example, using a drive motor 426.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A tube opening device comprising:
a body having a first side and a second side and including a tube-receiving cavity, the first side of the body including a first belt system disposed on a first side of the tube-receiving cavity and a second belt system disposed on a second side of the tube-receiving cavity, the first and second belt systems configured to apply pressure to a tube receive in the tube-receiving cavity.

2. The tube opening device of claim 1, wherein the first belt system includes a first belt overlaying at least a portion of a first belt drive and a first shaft in communication with the first belt drive, the first shaft being configured to rotate the first belt drive in a first direction and also in a second direction different form the first direction.

3. The tube opening device of claim 2, wherein the first belt system further includes a first support block disposed between the first belt drive and the tube-receiving cavity, the first support block being configured to maintain contact between the first belt and the tube as disposed in the tube-receiving cavity between the first and second belt systems.

4. The tube opening device of claim 3, wherein the first belt system further includes a second belt drive disposed on a first end of the first support block and a third belt drive disposed on an opposing second end of the first support block.

5. The tube opening device of claim 2, wherein the first shaft is configured to be moved in the first and second directions using an automatic operation.

6. The tube opening device of claim 2, wherein the first shaft is configured to be moved in the first and second directions using a manual operation.

7. The tube opening device of claim 2, wherein the second belt system includes a second belt overlaying at least a portion of a second belt drive and a second shaft in communication with the second belt drive, the second shaft being configured to rotate the second belt drive in a first direction and also in a second direction different form the first direction.

8. The tube opening device of claim 7, wherein the second belt system further includes a second support block disposed between the second belt drive and the tube-receiving cavity, the second support block being configured to maintain contact between the second belt and the tube as disposed in the tube-receiving cavity between the first and second belt systems.

9. The tube opening device of claim 8, wherein the second belt system further includes a third belt drive disposed on a first end of the second support block and a fourth belt drive disposed on an opposing second end of the second support block.

10. The tube opening device of claim 7, wherein the second shaft is configured to be moved in the first and second directions using an automatic operation.

11. The tube opening device of claim 7, wherein the second shaft is configured to be moved in the first and second directions using a manual operation.

12. A tube opening device comprising:
a body having a first side and a second side and including a tube-receiving cavity, the first side including two or more gears and the second side including a first belt system in communication with a first gear of the two or more gears and a second belt system in communication with a second gear of the two or more gears, the first belt system and the first gear being disposed on a first side of the tube-receiving cavity, and the second belt system and the second gear being disposed on a second side of the tube-receiving cavity.

13. The tube opening device of claim 12, wherein the first gear includes a first plurality of teeth, the second gear includes a second plurality of teeth, and the two or more gears further includes a third gear including a third plurality of teeth.

14. The tube opening device of claim 13, wherein the third gear is movable between a first position and a second position,
in the first position, the third plurality of teeth engage the first plurality of teeth of the first gear and the second plurality of teeth of the second gear, and
in the second position, the third plurality of teeth do not teeth engage the first plurality of teeth of the first gear or the second plurality of teeth of the second gear.

15. The tube opening device of claim 14, wherein the second side further includes a button configured to cause the third gear to move from the first position to the second position.

16. The tube opening device of claim 14, wherein in the first position, movement of the first gear is translated to the second gear and the first and second belts are moved in opposing directions.

17. The tube opening device of claim 16, wherein the first gear is configured to be moved by a first drive motor.

18. The tube opening device of claim 17, wherein in the second position, the second gear is configured to be moved by a second drive motor.

19. The tube opening device of claim 18, wherein the first drive motor is configured to move the first gear in a first direction and the second drive motor is configured to move the second gear in the first direction.

20. The tube opening device of claim 18, wherein the first drive motor is configured to move the first gear in a first direction and the second drive motor is configured to move the second gear in a second direction opposing the first direction.

21. The tube opening device of claim 12, wherein a first shaft couples the first gear and the first belt system and a second shaft couples the second gear and the second belt system, wherein the first and second shafts travel through the body.

22. The tube opening device of claim 21, wherein the first belt system includes one or more first pulleys and at least one of the one or more first pulleys is coupled to the first shaft.

23. The tube opening device of claim 22, wherein the second belt system includes one or more second pulleys and at least one of the one or more second pulleys is coupled to the second shaft.

24. The tube opening device of claim 12, wherein the first belt system includes one or more first pulleys and a first support block,
the one or more first pulleys including a first pulley disposed near a first end of the first support block and a second pulley disposed near a second end of the first support block,
the first support block being disposed adjacent to a first side of the tube-receiving cavity.

25. The tube opening device of claim 24, wherein the one or more first pulleys further include a third pulley disposed adjacent to the first support block away from the first side of the tube-receiving cavity.

26. The tube opening device of claim 24, wherein the second belt system include one or more second pulleys and a second support block,
the one or more second pulleys including a fourth pulley disposed near a first end of the second support block and a fifth pulley disposed near a second end of the second support block,
the second support block being disposed adjacent to a second side of the tube-receiving cavity, the second side of the tube-receiving cavity opposing the first side of the tube-receiving cavity.

27. The tube opening device of claim 26, wherein the one or more second pulleys further include a sixth pulley disposed adjacent to the second support block away from the second side of the tube-receiving cavity.
